(19) United States
(12) Patent Application Publication
(10) Pub. No.: US 2009/0286745 A1
(43) Pub. Date: Nov. 19, 2009

(54) INHIBITION OF ALPHA-SYNUCLEIN AGGREGATION

(75) Inventors: Jesus Zurdo, Cambridge (GB); Susan Fowler, Cambridge (GB); Yvette Stallwood, Cambridge (GB); Ernest Giralt, Barcelona (ES); Meritxell Teixido, Barcelona (ES); Natalia Carulla, Barcelona (ES)

Correspondence Address: KLARQUIST SPARKMAN, LLP, 121 SW SALMON STREET, SUITE 1600, PORTLAND, OR, 97204, US

(73) Assignee: Zapaloid Limited

(21) Appl. No.: 12/307,081

(22) PCT Filed: Jul. 2, 2007
(86) PCT No.: PCT/GB07/02469

§ 371 Date: Dec. 30, 2008

Related U.S. Application Data
(60) Provisional application No. 60/806,511, filed on Jul. 3, 2006.

(51) Int. Cl. A61K 38/08; C07K 7/06; C07K 5/10; A61K 38/07

(52) U.S. Cl. 514/15; 530/329; 530/330; 530/328; 514/16; 514/17

(57) ABSTRACT

This invention relates to the inhibition of alpha-synuclein aggregation using peptidyl compounds which are retroenantiomers of the alpha-synuclein sequence, in particular retroenantiomers of sequences in the regions between residues 1 to 60 or residues 61 to 96. Peptidyl compounds of the invention may optionally be coupled to doperminergic targeting moieties and/or blood brain barrier transport moieties and may be useful in the treatment of alpha-synucleinopathies such as Parkinson's disease.

This invention relates to the inhibition of protein aggregation, in particular the inhibition of α-synuclein aggregation. This may be useful, for example, in the treatment of α-synucleinopathies, such as Parkinson's disease.

Parkinson's disease (PD) is one of the major neurodegenerative disorders, affecting 3% of the population over the age of 65. It is characterized by resting tremor, bradykinesia, rigidity, and postural instability (Lang, A. E. & Lozano, A. M. N Engl J Med 339, 1044-53 (1998)). The primary pathological change in this disorder is a degeneration of dopaminergic neurons in the substantia nigra pars compacta (SNc) (Jenner, P. & Olanow, C. W. Ann Neurol 44, S72-84 (1998)). PD is the second most common neurodegenerative disorder, and its underlying disease mechanism(s) remains to be elucidated. The reason for this is partially due to the chronic nature of the disease itself (duration of 1-2 decades is common). The etiology of sporadic PD is still not fully understood but several potential contributing factors, including genetic aberrations, endogenous, and environmental factors have been proposed (Calne, D. Parkinsonism Relat. Disord. 7 3-7 (2000)).

The pathological hallmark lesion of PD is considered to be the deposition of fibrillar intracytoplasmic inclusions called the Lewy Bodies (LB), and this deposition may underlie the observed neurodegeneration. α-Synuclein (α-SN) is a ubiquitous 140-amino acid protein of 18-20 kDa which is abundant in neurons, especially in presynaptic terminal (Goedert, M. Nat Rev Neurosci 2, 492-501 (2001), Iwatsubo, T. J Neurol 250 Suppl 3, III11-4 (2003)), and is also found to be the major protein component in LBs (Spillantini, M. G. et al. Nature 388, 839-40 (1997). Lewy pathology is also characteristic for dementia with LBs (DLB), the LB variant of AD, and neurodegeneration with iron-accumulation type I (Hallervorden-Spatz disease). Moreover, α-SN fibrils are deposited in (oligodendro) glial cytoplasmic inclusions (GCIs) of patients with multiple system atrophy. These disorders are commonly referred to as the α-synucleinopathies (Spillantini, M. G et al Ann N Y Acad Sci 920, 16-27 (2000), Golbe, L. I. Mov Disord 14, 6-9 (1999), Goedert, M. et al. Biochem Soc Trans 26, 463-71 (1998), Goedert, M. et al Mol Psychiatry 3, 462-5 (1998))

Further evidence supporting the importance of α-SN in PD is the linkage of genetic alterations involving α-SN with several early onset forms of PD. These mutations include A30P, A53T, E46K and α-SN trisomy (Vila, M. et al Nat Med 10 Suppl, S58-62 (2004), Farrer, M. et al. Ann Neurol 55, 174-9 (2004), Zarranz, J. J. et al. Ann Neurol 55, 164-73 (2004)).

Genetic alteration affecting other proteins, particularly components of the Ubiquitine-Proteasome system (UPS), seems to be the origin of several forms of familial PD.

At present, there is no effective treatment for PD, and only symptomatic treatments are available (e.g. levodopa and dopaminergic agonists).

Several new approaches are currently under investigation for the treatment of PD. Regeneration of the substantia nigra has been attempted using stem cell therapy to replace cells lost during the earlier stages of the disease (Lindvall, O. et al Nat Med 10 Suppl, S42-50 (2004)). However, no significant success has been achieved at present. Gene therapy (in some cases used in combination with stem cells) has been attempted to replace the biosynthetic enzymes involved in dopamine synthesis or add neurotrophic factors for protection and restoration of dopaminergic neurons (GDNF) (Behrstock, S. et al Ann N Y Acad Sci 1019, 5-14 (2004), Azzouz, M. et al. Neuroreport 15, 985-90 (2004), Fraix, V. Rev Med Interne 25, 524-7 (2004)). Some trials involving neurotrophic factors have been initiated (Oransky, I. Lancet 362, 712 (2003), Howard, K. Nat Biotechnol 21, 1117-8 (2003)). Inhibition of α-SN aggregation has also been attempted using peptidyl inhibitors (Bodles, A. M. et al Neurosci Lett 359, 89-93 (2004)), for example based on β-SN (Windisch, M. et al. J Mol Neurosci 19, 63-9 (2002), Park, J. Y. et al, Biochemistry 42, 3696-700 (2003), Windisch, M. et al J Mol Neurosci 24, 155-66 (2004)), or the NAC region of α-SN itself (El-Agnaf, O. M. et al. Faseb J 18, 1315-7 (2004)). However, inhibition has only been shown at high concentrations, the data from the reported cell-death inhibition experiments are difficult to interpret and the cell models and assays employed are not necessarily relevant to PD. Further problems arise from the degradation of peptide inhibitors in plasma. Single chain antibodies have been reported to interfere with the fibrillation of α-SN and to delay the formation of early oligomers in vitro (Emadi, S. et al. Biochemistry 43, 2871-8 (2004)). A similar approach has been tried in Huntington's disease (involving Huntingtin aggregation). In this latter case, a single-domain V_{L} intrabody (intra-cellular antibody) seemed to inhibit huntingtin aggregation in mammalian cells (Colby, D. W. et al. J Mol Biol 342, 901-12 (2004)). Although there is no example of a commercial drug using intrabodies, some clinical trials have been initiated using intrabodies as an additional tool in gene therapy (Alvarez, R. D. et al. Clin Cancer Res 6, 3081-7 (2000)).

The present inventors have identified retroenantiomers of particular regions of α-synuclein which are active in inhibiting the aggregation of α-synuclein and may therefore be useful in the treatment of PD and other α-synucleinopathies.

One aspect of the invention provides a peptide or other peptidyl compound consisting of four to ten D-amino acids having the reverse sequence of a contiguous amino acid sequence within the region between residues 1-96 of α-synuclein.

A peptide described herein may inhibit the aggregation of α-synuclein and may, for example, consist of 4, 5, 6, 7, 8, 9 or 10 D-amino acids, preferably 6, 7 or 8 D-amino acids.

In some preferred embodiments, a peptide may consist of four to ten D-amino acids having the reverse sequence of a contiguous amino acid sequence in the region between residues 1-60 of α-synuclein. In other words, the D-amino acid sequence in the N terminal to C terminal direction corresponds to the contiguous amino acid sequence of α-synuclein in the C terminal to N terminal direction. A D-amino acid sequence which is the reverse of an L-amino acid sequence is commonly known as a ‘retroenantiomer’ of that sequence.

For example, a peptide may consist of the reverse sequence of a contiguous amino acid sequence which comprises one or more, two or more, three or more, four or more, five or more, six or more, seven or more, or all the residues from the region between residues 1-7, 14-20, 36-42 or 47-57 of α-synuclein. In some preferred embodiments, a peptide may consist of a sequence of D-amino acids selected from the group consisting of: gkmfvdm, sgvylvg, and vtavghv.

In other embodiments, a peptide may consist of a sequence of four to ten D-amino acids which is the reverse of a contiguous amino acid sequence in the region between residues 61 to 96 of α-synuclein. For example, a peptide may consist of the reverse sequence of a contiguous amino acid sequence which comprises one or more, two or more, three or more, four or more, five or more, six or more, seven or more, eight or more, nine or more or all ten residues from the region between residues 64 to 76 or 86 to 96 of α-synuclein. In some preferred embodiments, a peptide may consist of a sequence of D-amino acids selected from the group consisting of: tvvaggv, atvgtvv, taaaisg, fgtaaai and kvfgtaa.

In some embodiments, a peptide may not consist of the reverse sequence of a contiguous amino acid sequence which comprises one, two, three, or four residues from the region between residues 69-72 (e.g. TVVA or VVA) of α-synuclein.

In some embodiments, a peptide may not consist of the reverse sequence of a contiguous amino acid sequence which comprises one, two or three residues from the region between residues 76-78 (AVA) of α-synuclein.

In some embodiments, a peptide may not consist of the reverse sequence of a contiguous amino acid sequence which comprises one, two, three or four residues from the region between residues 88-91 (AAAI) of α-synuclein.

The present inventors have also found that peptides which are not retroenantiomers of α-synuclein may also interact with α-synuclein and reduce or inhibit aggregation.

Another aspect of the invention provides a peptide consisting of four to ten D-amino acids which interacts with a region of α-synuclein between residues 1-96, for example, a region between residues 1-60 of α-synuclein or a region between residues 61 to 96 of α-synuclein.

The peptide preferably binds to the region of α-synuclein, for example through the formation of hydrogen bonds to form a β-sheet secondary structure with the amino acids of the region of α-synuclein.

A suitable peptide may inhibit the aggregation of α-synuclein and may, for example, consist of 4, 5, 6, 7, 8, 9 or 10 D-amino acids, preferably 6, 7 or 8 D-amino acids, as described above.

In some embodiments, a peptide may consist of a sequence of D-amino acids which interacts with a region of α-synuclein between residues 61-66 (EQVTN). A suitable peptide may comprise or consist of the D-amino acid sequence qysvli (ZP-0195) or may comprise or consist of the D-amino acid sequence qysvli with one, two or three amino acid substitutions. For example, a peptide may consist of a D-amino acid sequence selected from the group consisting of: qykyli, qysvpi, qyspli, qypvli, rysvli, qysvli, qytyli, pysvli, or qysvlv.

Suitable peptides may be designed using any convenient method. Some suitable computer-based methods are described in co-pending patent application U.S. 60/821,553.

A peptide may comprise one, two or three additional N terminal residues.

For example, a peptide may comprise or consist of a sequence selected from the group consisting of: ekysvli and drysvli.

In other embodiments, a peptide may consist of a sequence of D-amino acids which interacts with a region of α-synuclein between residues 71-76 (VTGVT). For example, a peptide may consist of the D-amino acid sequence of hhviva (ZP-0158) or may comprise or consist of the D-amino acid sequence hhviva with one, two or three amino acid substitutions. Preferably, the N-terminal histidine residues are not substituted.

For example, a peptide may comprise or consist of a sequence selected from the group consisting of: hhvvva, hhylva, hhvkva, hhveva, hpviva, hhvivp, hhvivv, hhvivt, hhvivy, hhvivw, hhtivv, hhtivk, hhtvva, hhtlva, hhtlvv, hhtevy and hhttvy.

The present inventors have also found that peptides which stabilise the secondary helical structure of α-synuclein reduce or inhibit aggregation. Suitable peptides interact with regions of α-synuclein sequence which have a propensity to form a helix or turn, and stabilize secondary structure in that region. These regions may be identified using known protein analysis algorithms, such as AGADIR (Muñoz, V. & Serrano, L. (1994) Nature: Struct. Biol. 1, 399-409; Muñoz, V. & Serrano, L. (1994) J. Mol. Biol. 245, 275-296; Muñoz, V. & Serrano, L. (1994) J. Mol. Biol. 245, 297-308; Muñoz, V. & Serrano, L. (1997) Biopolymers 41, 495-509; Lacroix, E. et al (1998) J. Mol. Biol. 284, 173-191).

Another aspect of the invention provides a peptide which interacts with α-synuclein and consists of the D-amino acid sequence kgegk, rdr, egkgegk, or rgdgd.

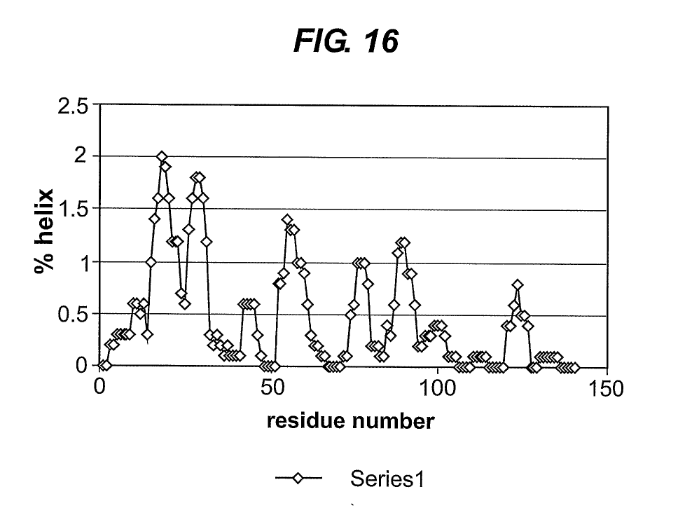

Preferably, such a peptide interacts with a region with α-helical propensity, for example residues 15-23, 26-31, 52-62, 74-79 or 87-93, as shown in FIG. 16 (see Ulmer et al (2005) J. Biol. Chem. 280 9595-903).

Peptides of the invention also encompass sequences which consist of an amino acid sequence set out herein with 1, 2, 3 or 4 D-amino acids added, deleted or substituted.

For example, 1, 2, 3 or 4 D-amino acids may be added or deleted from the N-terminal or C-terminal of a peptide sequence set out herein.

The 1, 2, 3 or 4 additional D-amino acids which are added to a peptide set out herein may be the reverse sequence of amino acids which adjoin the N-terminal or C-terminal of the contiguous sequence in α-synuclein. Alternatively, the 1, 2, 3 or 4 additional D-amino acids may be residues which are not the reverse sequence of amino acids which adjoin the N-terminal or C-terminal of the contiguous sequence in α-synuclein (i.e. they may be heterologous amino acids). In some embodiments, one or more N-methyl-phenylalanine residues may be added to the N terminal, as described below, to facilitate transport across the blood brain barrier.

A substitution may be a conservative or non-conservative substitution. For example, a peptide may consist of sequences having one, two, three or more conservative or non-conservative substitutions relative to a sequence set out herein. A conservative substitution is a replacement of a D-amino acid residue with another of similar properties, such as charge, polarity and/or hydrophobicity. For example, conservative substitutes for an amino acid within the native polypeptide sequence can be selected from other members of the class to which the amino acid belongs. Amino acids can be divided into the following four groups: (1) acidic amino acids, (2) basic amino acids, (3) neutral polar amino acids, and (4) neutral, nonpolar amino acids. Representative amino acids within these various groups include, but are not limited to, (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, cystine, tyrosine, asparagine, and glutamine; and (4) neutral nonpolar (hydrophobic) amino acids such as alanine, leucine, isoleucine, valine, proline, phenylalanine, tryptophan, and methionine. Conservative substitution tables listing functionally similar amino acids are known in the art (Altschul, S. F. 1991 Journal of Molecular Biology 219: 555-665, Crighton (1984) Proteins, W. H. Freeman and Company). For example, a peptide consisting of a sequence having one, two, three or more conservative substitutions may show 95%, 99% or 100% sequence similarity to a sequence set out herein. Amino acid similarity may be defined with reference to the algorithm GAP (Accelerys), or the TBLASTN program, of Altschul et al. (1990) J. Mol. Biol. 215: 403-10.

A peptide may consist of a sequence set out herein with one, two, three or more substitutions which reduce or prevent beta strand association. For example, one or more, 2 or more, 3 or more or 4 D-amino acids in the peptide sequence may be replaced by D-proline. In some embodiments, the residue at position 2 and/or position 3 may be replaced by D-proline. For example, one or more D-amino acids in the reverse sequence of a contiguous amino acid sequence in the region between residues 1-60 of α-synuclein, or in the region between residues 61 to 96 region of α-synuclein, may be replaced by D-proline. Examples of such peptides include peptides consisting of a D-amino acid sequence selected from the group consisting of: gapevtk, tpaaisg, tapaisg, fptaaai, kpfgtaa, kvfptaa, sptvvags, and gpvntvq. In some preferred embodiments, a peptide may consist of the D-amino acid sequence gpvntvq.

Peptides of the invention also encompass sequences which consist of a sequence set out herein with 1, 2, 3 or 4 modified D-amino acids. D-amino acids in peptides described herein may be modified, for example by the introduction of a substituent chemical group, for example at the N position. Suitable substituent groups include halogens such as F, nitrate, and alkyl groups, such as methyl or acetyl groups.

An amino acid modification may reduce or prevent beta strand association. For example, one or more, for example 2, 3 or 4 D-amino acids in the peptide sequence may be N-substituted, preferably N-alkylated, for example N-methylated or N acetylated.

Peptides may be generated wholly or partly by chemical synthesis. D-amino acid peptides, such as retroenantiomers, may be produced by employing D-form derivatized amino acid residues in the chemical synthesis. Suitable D-amino acids for solid phase peptide synthesis are commercially available (e.g., Advanced Chem Tech, Louisville; Nova Biochem, San Diego; Sigma, St Louis; Bachem California Inc., Torrance, etc.). The peptides can be readily prepared, for example, according to well-established, standard liquid or, preferably, solid-phase peptide synthesis methods, general descriptions of which are broadly available (see, for example, in J. M. Stewart and J. D. Young, Solid Phase Peptide Synthesis, 2nd edition, Pierce Chemical Company, Rockford, Ill. (1984), in M. Bodanzsky and A. Bodanzsky, The Practice of Peptide Synthesis, Springer Verlag, New York (1984); in J. H. Jones, The Chemical Synthesis of Peptides. Oxford University Press, Oxford 1991; in Applied Biosystems 430A Users Manual, ABI Inc., Foster City, Calif., in G. A. Grant, (Ed.) Synthetic Peptides, A User's Guide. W. H. Freeman & Co., New York 1992, E. Atherton and R. C. Sheppard, Solid Phase Peptide Synthesis, A Practical Approach. IRL Press 1989 and in G. B. Fields, (Ed.) Solid-Phase Peptide Synthesis (Methods in Enzymology Vol. 289). Academic Press, New York and London 1997), or they may be prepared in solution, by the liquid phase method or by any combination of solid-phase, liquid phase and solution chemistry, e.g. by first completing the respective peptide portion and then, if desired and appropriate, after removal of any protecting groups being present, by introduction of the residue X by reaction of the respective carbonic or sulfonic acid or a reactive derivative thereof.

Peptides as described above may be fused to one or more sequences which are not retroenantiomers of α-synuclein sequences. Peptides and oligopeptides comprising peptides as described above are also provided as aspects of the present invention, particularly wherein the peptide is fused to one or more sequences which are not retroenantiomers of α-synuclein sequences (i.e. heterologous sequences).

By “heterologous” is meant not being the retro-enantiomer of a natural α-synuclein sequence which is joined by a peptide bond without intervening amino acids to the contiguous α-synuclein sequence described herein. Usually, where heterologous amino acids are fused to the peptide, the whole contiguous sequence of amino acids does not occur within α-synuclein, and may be 10 or more, preferably 15 or more, more preferably 20 or more, 25 or more or 30 or more amino acids. Heterologous sequences of amino acids which may be fused to a peptide described herein may include antibodies or antibody fragments, such as Fabs, F(ab′)₂s, dAbs, Fvs, and scFvs, neurotrophins such as NGF BDNF, NT3, and GDNF, Insulin-like Growth Factors, such as IGF1 and IGF2, transferrin and other peptides that bind to the transferrin receptor, and other coupling partners involved in BBB transport or dopaminergic neuron transport, as described herein.

Peptides and oligopeptides as described herein may be N-terminal and/or C-terminal modified, for example by addition of a coupling partner or moiety. Coupling partners which may be linked to a peptide may include protecting groups, for example to help to increase the half-life of the peptide in vivo, and targeting groups.

Suitable protecting groups are well-known in the art (e.g., Greene e al., (1991) Protective Groups in Organic Synthesis, 2nd ed., John Wiley & Sons, Inc. Somerset, N.J.) and include acetyl, amide, and 3 to 20 carbon alkyl groups, Fmoc, t-boc, 9-fluoreneacetyl group, 1-fluorenecarboxylic group, 9-florenecarboxylic group, 9-fluorenone-1-carboxylic group, benzyloxycarbonyl, Xanthyl (Xan), Trityl (Trt), 4-methyltrityl (Mtt), 4-methoxytrityl (Mmt), 4-methoxy-2,3,6-trimethylbenzenesulphonyl (Mtr), Mesitylene-2-sulphonyl (Mts), 4,4-dimethoxybenzhydryl (Mbh), Tosyl (Tos), 2,2,5,7,8-pentamethyl chroman-6-sulphonyl (Pmc), 4-methylbenzyl (MeBzl), 4-methoxybenzyl (MeOBzl), Benzyloxy (BzlO), Benzyl (Bzl), Benzoyl (Bz), 3-nitro-2-pyridinesulphenyl (Npys), 1-(4,4-dimentyl-2,6-diaxocyclohexylidene)ethyl (Dde), 2,6-dichlorobenzyl (2,6-DiCl-Bzl), 2-chlorobenzyloxycarbonyl (2-Cl-Z),2-bromobenzyloxycarbonyl (2-Br-Z), Benzyloxymethyl (Bom), t-butoxycarbonyl (Boc), cyclohexyloxy (cHxO),t-butoxymethyl (Bum), t-butoxy (tBuO), t-Butyl (tBu), Trifluoroacetyl (TFA), Caffeic acid, formyl-, biotin and carboxyfluorescein.

In some embodiments, an acetyl group may be used to protect the amino terminus and/or an amide group may be used to protect the carboxyl terminus. Acetylation may, for example, be accomplished during the synthesis when the peptide is on the resin using acetic anhydride. Amide protection may, for example, be achieved by the selection of a proper resin for the synthesis.

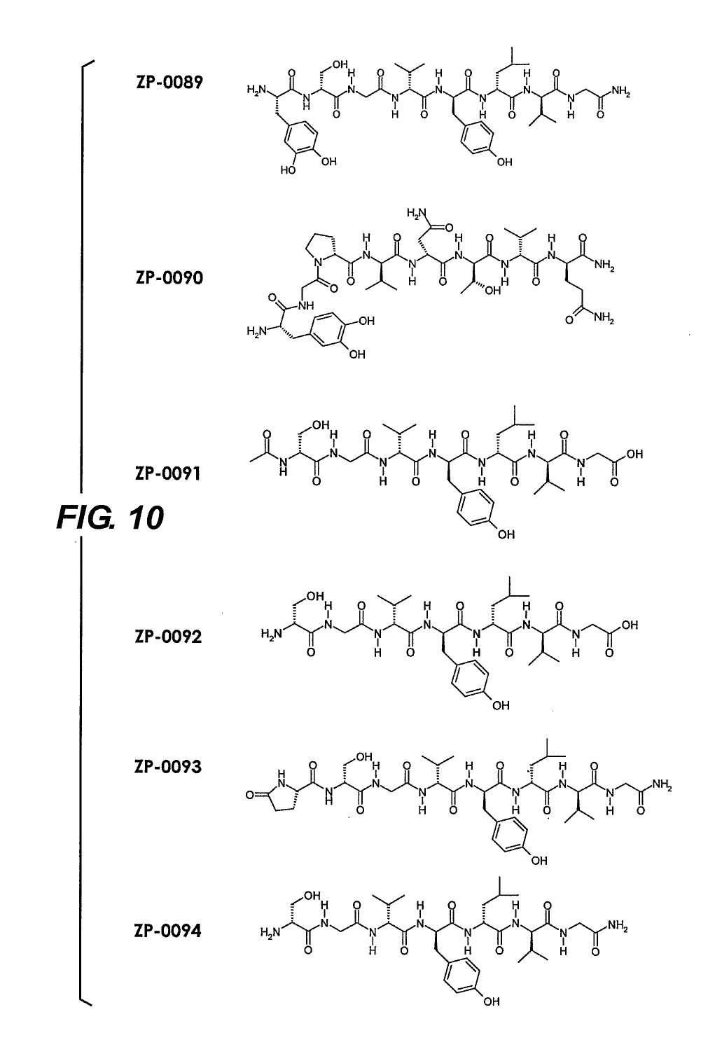

Examples of peptides described herein which are linked to protecting groups include ZP0091, ZP0092 and ZP0094, which are shown in FIG. 10.

Suitable targeting groups which may be linked to a peptide include dopaminergic neuron targeting moieties, which may, for example, be attached to the N or C terminal of the peptide sequence. Suitable dopaminergic neuron targeting moieties include dopamine analogues, such as L-DOPA, DOPA agonists (Sever et al Tetehedron 2001 57 6139; Appell et al Biochem Phamacol 2004 67 293), pyroglutamic acid, transferrin and SAP.

Examples of peptides described herein which are linked to dopaminergic neuron targeting moieties include ZP0089, ZP0090 and ZP0093 shown in FIG. 10.

Other coupling partners include Blood Brain Barrier (BBB) transport moieties, which may, for example be attached to the N or C terminal of the peptide sequence. A Blood Brain Barrier (BBB) transport moiety may include moieties which facilitate passive diffusion across the BBB and moieties that interact with a receptor or carrier and cross the BBB by receptor or carrier mediated endocytosis, such as Sweet Arrow Peptide (SAP) (Fernandez-Carneado et al Angew Chem Int Ed Engl 2004 43 14 1811-1814) and retroviral TAT protein (C. Foerg et al Biochemistry 2005 44 72). Suitable Blood Brain Barrier (BBB) transport moieties include N-methyl phenylalanine (NMePhe) which has been shown to enhance transport across the BBB (Conradi, R. A. et al Pharm. Res. (1992) 9, 435-439; Chikhale, E. G. et al Pharm. Res. 1994, 11, 412-419; Chikhale, E. G. et al J. Pharmacol. Exp. Ther. (1995) 273, 298-303), transferrin, IGF1, IGF2 and leptin. Peptides may be synthesised with one or more, for example 1, 2, 3 or 4 N-methyl phenylalanine residues using standard synthesis techniques.

Techniques for coupling peptides to both peptidyl and non-peptidyl coupling partners are well-known in the art.

A compound comprising a peptide as described herein linked to one or more coupling partners is provided by another aspect of the invention.

A peptide or compound as described herein may be used in a method of treatment of the human or animal body, for example for use in the treatment of an α-synucleinopathy, or in the manufacture of a medicament for the treatment an of α-synucleinopathy.

A method of treatment of α-synucleinopathy may comprise;
administering a peptide as described herein as described herein to an individual in need thereof.

α-Synucleinopathies are conditions associated with the aggregation of α-synuclein and include Parkinson's disease, LB variant Alzheimer's disease, multiple system atrophy (MSA), LB dementia and Hallervorden-Spatz disease.

Administration of a peptide or compound described herein is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors.

A peptide or compound as described herein described herein may be administered as a pharmaceutical composition. A pharmaceutical composition may include, in addition to the peptide or compound, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, nasal or by injection, e.g. cutaneous, subcutaneous or intravenous.

Another aspect of the invention provides a method of producing a pharmaceutical composition, for example for use in treating α-synucleinopathy, comprising;
admixing a peptide or compound described herein with a pharmaceutically acceptable excipient, carrier, buffer or stabiliser.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

Pharmaceutical compositions suitable for nasal administration, wherein the carrier is a solid, include a coarse powder having a particle size, for example, in the range of about 20 to about 500 microns which is administered in the manner in which snuff is taken, i.e. by rapid inhalation through the nasal passage from a container of the powder held close up to the nose. Suitable formulations wherein the carrier is a liquid for administration as, for example, nasal spray, nasal drops, or by aerosol administration by nebuliser, include aqueous or oily solutions of the active compound.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, or Lactated Ringer's Injection. Preservatives, stabilisers, buffers, antioxidants and/or other additives may be included, as required.

A composition comprising a peptide or compound described herein may be administered alone or in combination with other treatments, either simultaneously or sequentially dependent upon the condition to be treated.

Controls are employed as appropriate within the routine knowledge and expectation of those skilled in the art.

Various further aspects and embodiments of the present invention will be apparent to those skilled in the art in view of the present disclosure, including the following experimentation to illustrate embodiments of the invention and the accompanying figures.

All documents mentioned in this specification are incorporated herein by reference in their entirety.

The term “comprises” as used herein implies the inclusion of a stated integer or group of integers but not the exclusion of any other integer or group of integers. The term “comprises” encompasses embodiments in which the stated integer or group of integers is included and any other integer or group of integers is excluded and may be replaced by ‘consists of’ when referring to such embodiments.

All peptide structures and sequences are indicated using the standard amino acid single letter code.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

Certain aspects and embodiments of the invention will now be illustrated by way of example and with reference to the figures described below.

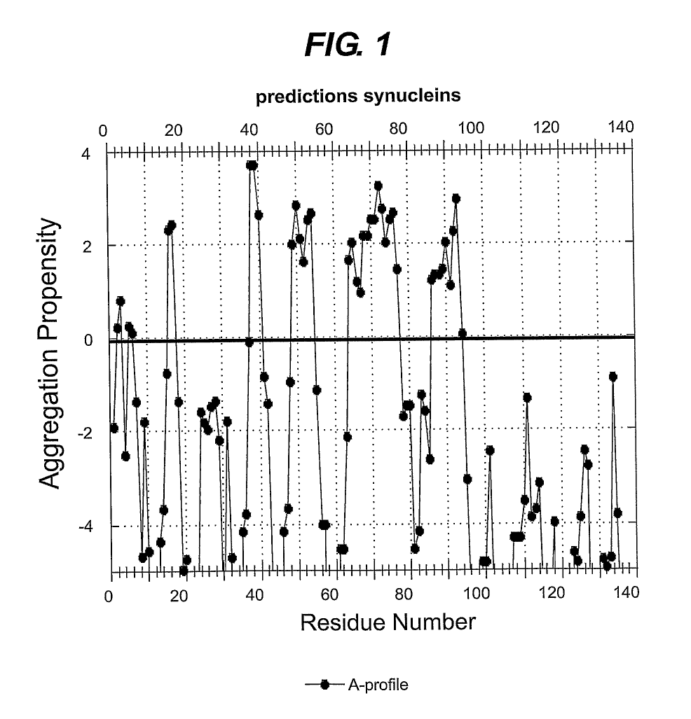

FIG. 1 shows an aggregation profile for alpha-synuclein calculated using methods described in refs 1-3 where the calculated aggregation propensity for each residue is plotted. This plot shows that regions with the highest aggregation propensity are in the regions of sequence from residues 1-7, 14-20, 36-42 and 61-95 (also known as the NAC region).

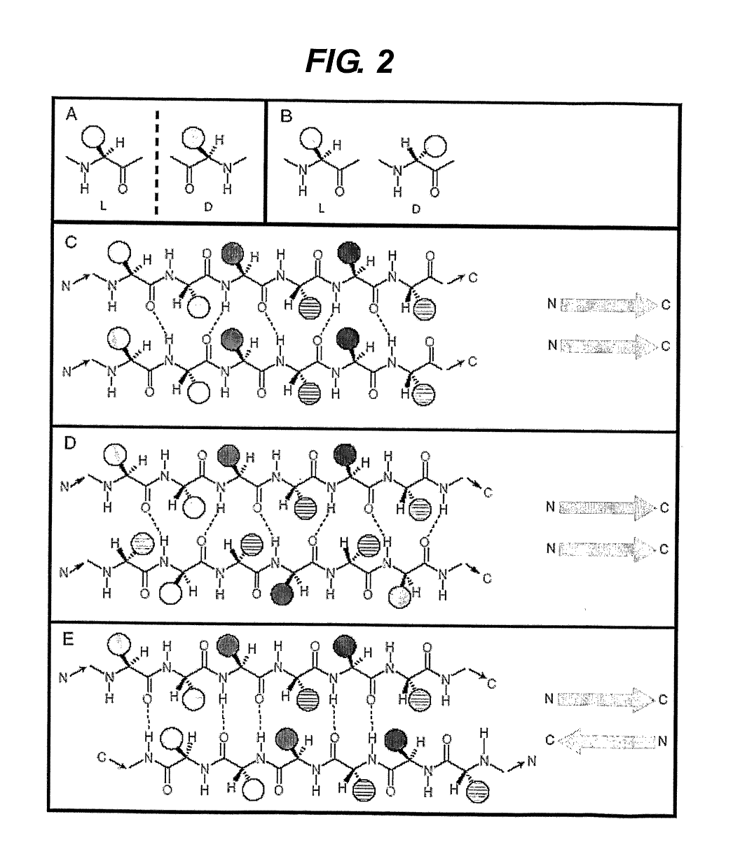

FIG. 2 shows the retro-enantio approach. A and B show a comparison of side chain orientation between L- and D amino acids. C shows a strand of parallel beta sheet of L-amino acids where side chains denoted by filled circles are interacting above the sheet and striped ones are interacting below the sheet. When one of the interacting sheets is replaced by D-amino acids (D), the orientation of the side chains changes such that those that were above the sheet are now below and thus the interactions seen in C are not maintained. By using D-amino acids in the reverse sequence (retro-enantio) the correct side chain interactions may be now be maintained in an anti-parallel beta sheet arrangement.

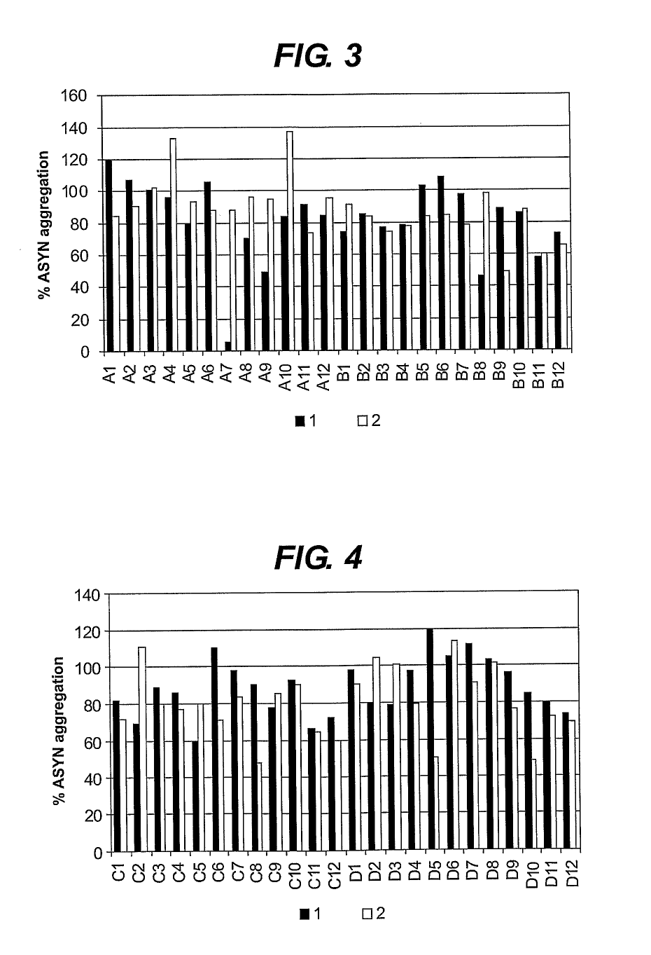

FIGS. 3 to 6 show graphs depicting the extent of aggregation undergone by ASYN in the presence of inhibitor. Endpoint ThT fluorescence was measured and expressed as a percentage of the endpoint aggregation measured for ASYN alone. Results from PBS and PEG experiments are presented on the same graph for comparison. Samples are named by plate position and their sequences can be referred to in Table 1.

FIG. 3 shows data for A1 to B12 (ZP-0001 to ZP-0024).

FIG. 4 shows data for C1 to D12 (ZP-0025 to ZP-0048)

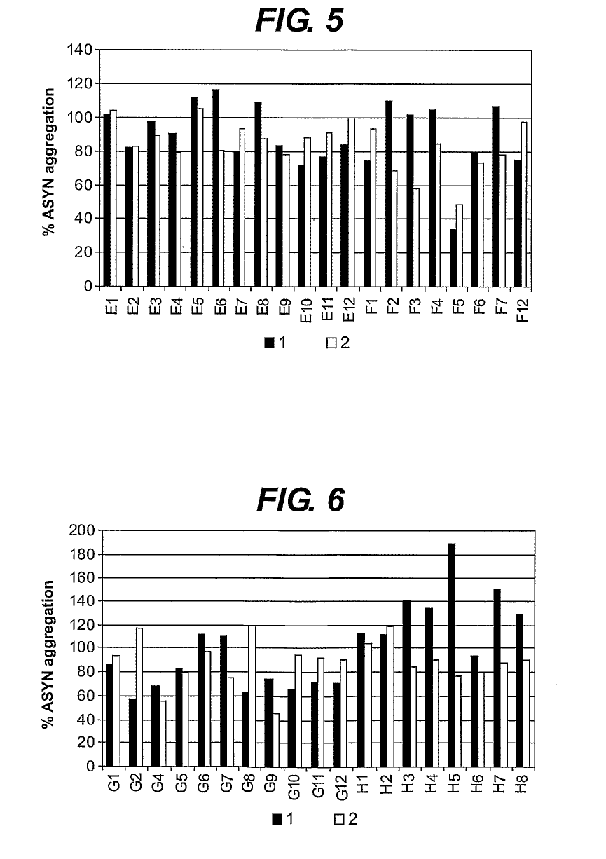

FIG. 5 shows data for E1 to F12 (ZP-0049 to ZP-0068) and

FIG. 6 shows data for G1 to H12 (ZP-0069 to ZP-0088).

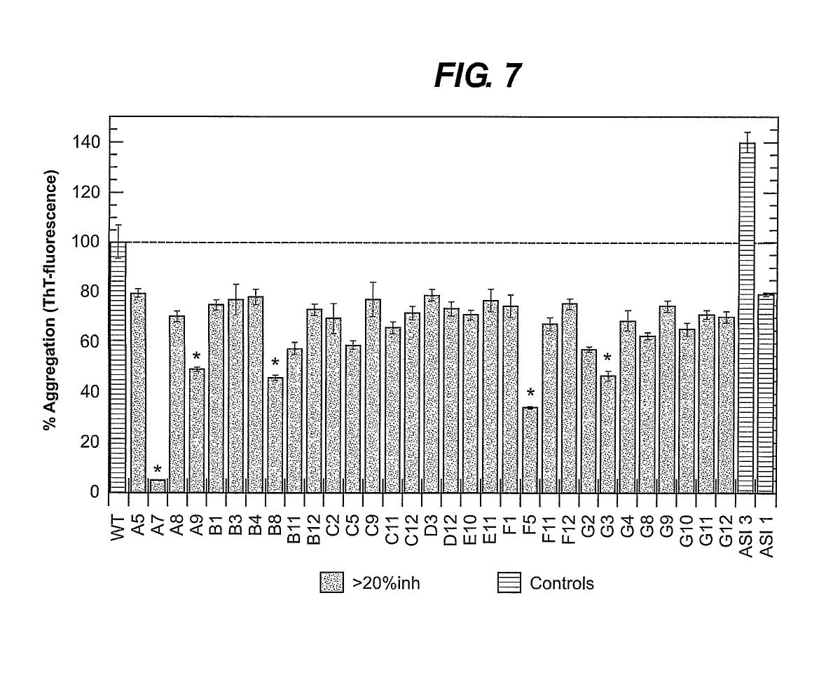

FIG. 7 shows a selection of the best performing inhibitors from the ASYN aggregation in PBS. Graphs depicting the extent of aggregation undergone by ASYN in the presence of inhibitor. Endpoint ThT fluorescence was measured and expressed as a percentage of the endpoint aggregation measured for ASYN alone.

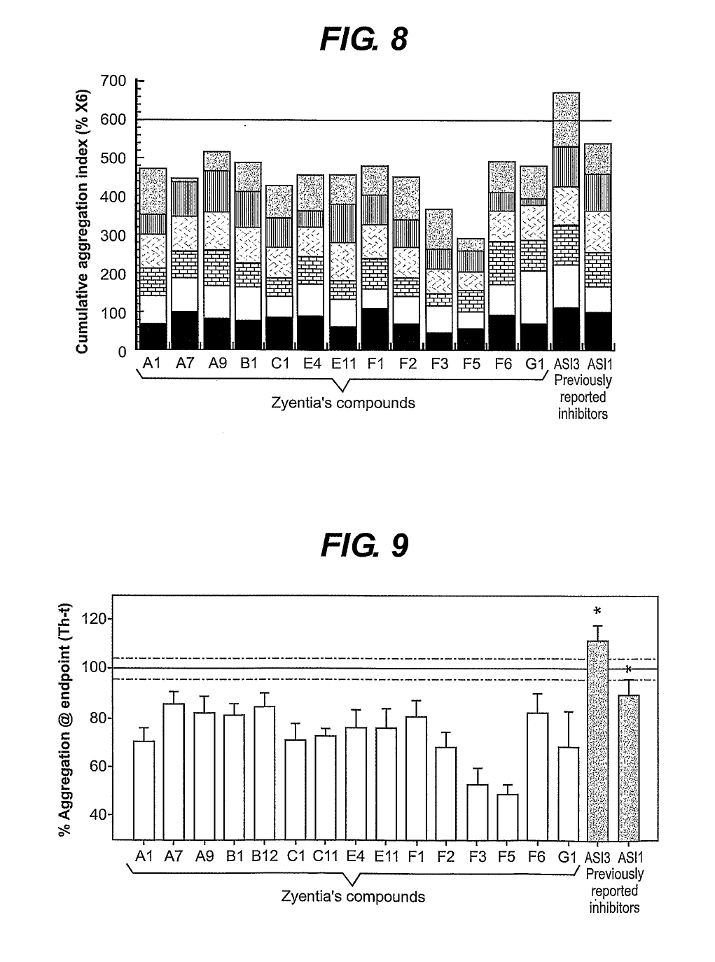

FIG. 8 shows inhibition of α-synuclein aggregation by a series of compounds. Data correspond to the sum of the aggregation endpoint values measured as a percentage of the aggregation undergone by alpha synuclein alone for 6 separate experiments. For inhibitor sequences see Table 1.

FIG. 9 shows the inhibition of α-synuclein by a series of compounds. Data refer to the percentage of aggregation undergone by alpha-synuclein alone. Bars reflect the averaged aggregation±standard error values measured under six different experimental conditions. A reference has been included corresponding to the value measured for α-synuclein alone±standard error.

FIG. 10 shows the chemical structures of peptides listed in Table 3.

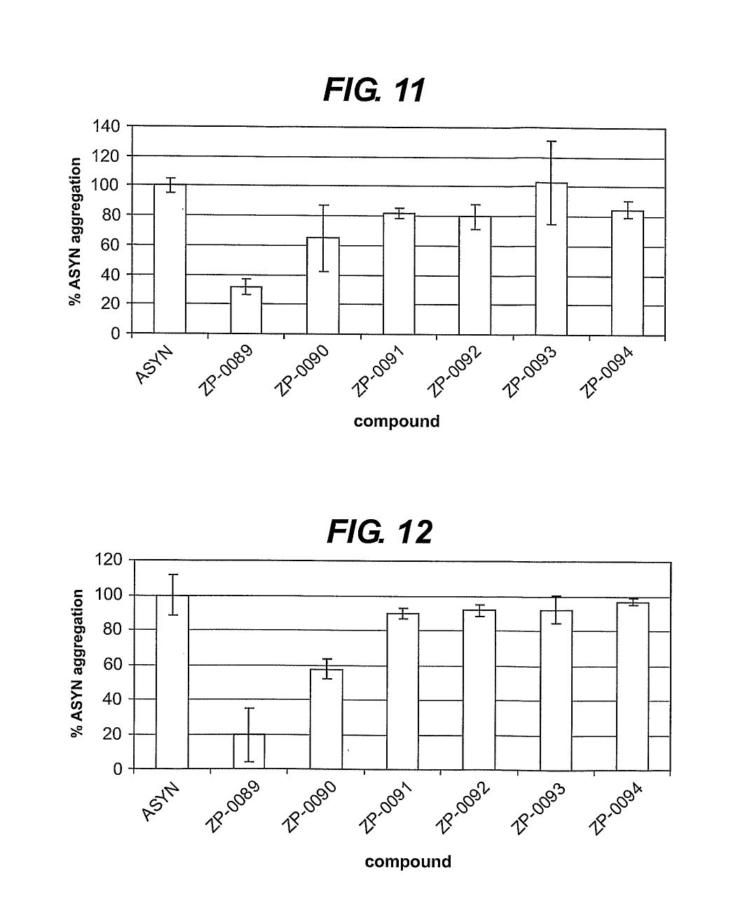

FIGS. 11 and 12 show endpoint thioflavin T measurements for ASYN in the presence of inhibitors ZP-0089 to ZP-0094 (Table 3 and FIG. 6). Each graph shows the average of 3 measurements with the standard deviation and is expressed as a percentage of the measurement for ASYN alone.

FIG. 11 shows results from an aggregation assay performed in Tris-Cl pH 7.4 at 37° C. with 100 mg/ml PEG 3350. ZP-0089 and ZP-0090 show a significantly decreased ThT signal compared to ASYN.

FIG. 12 shows results from an aggregation assay performed in Tris-Cl pH 7.4 at 37° C. with 100 mg/ml PEG 3350. ZP-0089 and ZP-0090 show a significantly decreased ThT signal compared to ASYN

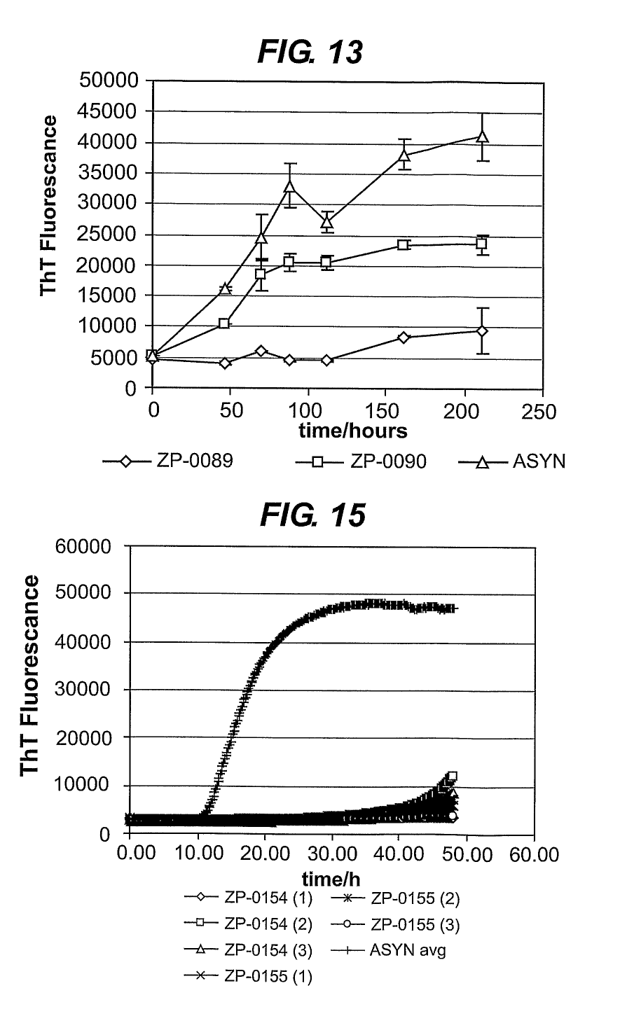

FIG. 13 shows aggregation kinetics of α-synuclein alone with PBS buffer in the presence of ZP-0089 and ZP-0090 (L-DOPA-A7 and L-DOPA-B8). The molar ratio of asyn:inhibitor is 1:2. For both, ASYN aggregation inhibition is observed as an increased lag time along with decreased endpoint ThT fluorescence.

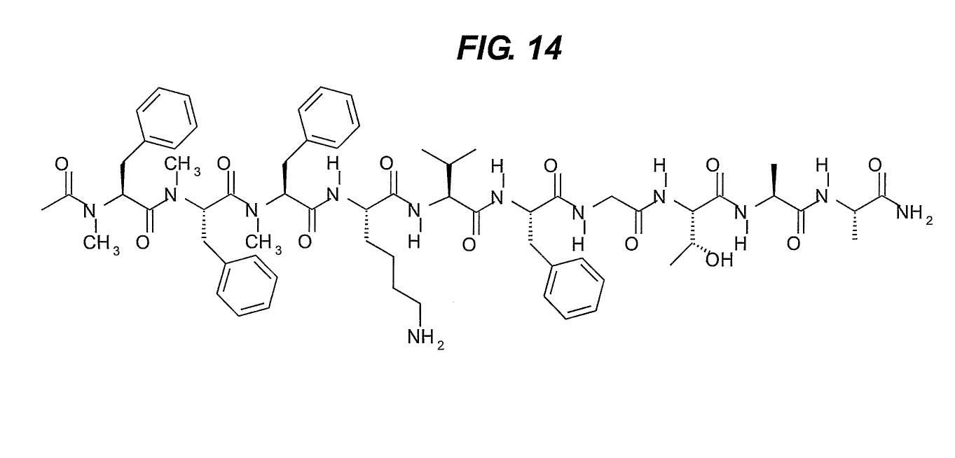

FIG. 14 shows the chemical structure of ZP-0155.

FIG. 15 shows the inhibition of ASYN aggregation by ZP-0154 and ZP-0155

FIG. 16 shows the regions of sequence in α-synuclein with a higher propensity to be in an alpha helical conformation. Values calculated using Agadir at pH 7.4 and 25° C.

### EXPERIMENTS

### Materials & Methods

10× PBS was supplied by Gibco at pH 7.4, Thioflavin T, tris buffers and PEG 3350 were supplied by Sigma.

### Peptide Synthesis

All peptide libraries were synthesized by Alta Biosciences (Birmingham, UK) using their Episcan array method. The 96 peptides in the library were synthesized on a 2 μmol scale and supplied lyophilized in 96-well plate format. Each sample in the library was dissolved in 2 ml 50% acetonitrile and 0.1 ml of each peptide sub-aliquoted into 20 plates and was lyophilized. Each plate contained 100 nmol of each peptide. The purity of these peptides was unknown. The plates were sealed and stored at −80° C. until required. (See Table 1, ZP-0001 to ZP-0087 for sequence details)

The group of peptides termed the ‘Giralt series’ were synthesized by standard Fmoc synthesis and purified by reverse phase HPLC. See Table 3 for sequence details, ZP-0089 to ZP-0094.

Several of the inhibitors from the Barcelona library that proved positive for ASYN aggregation inhibition were synthesized by Alta Biosciences on a 5 μmol scale and purified using reverse phase HPLC. Their identity was confirmed using MALDI mass spectrometry and their purity was >80%.

### Endpoint Thioflavin T Assay.

Thioflavin T has been used extensively to report on the aggregation state of proteins and associates rapidly with amyloid fibrils, giving rise to an increase in fluorescence intensity at 482 nm (Le Vine, H. (1993) Protein Sci. 2, 404-410). It can thus be used to quantify the amount of conversion of a given peptide into amyloid. A 2.5 mM thioflavin T (ThT) stock was prepared in the same buffer that the assay aggregation was performed in and filtered through a 0.22 μM Millex GV filter from Millipore. This stock was further diluted and added to the aggregated samples taken to give a final ThT concentration of 62.5 μM. Fluorescence was measured in a Biotek Synergy HT reader with cut off filters for excitation at 440/30 nm and emission at 485/20 nm.

### Kinetic Thioflavin T Assay.

Kinetic ThT assays were performed by adding ThT directly to the sample at time=0. A 2.5 mM ThT stock solution was made up in the buffer that was used in the aggregation experiment, filtered through a 0.22 μM Millex GV filter and diluted to 62.5 μM by adding 5 μl to final sample volume of 200 μl in a 96-well polypropylene plate. The increase in fluorescence over time as a result of amyloid formation was measured in a Biotek Synergy HT plate reader at 37° C. with shaking. Cut off filters were used for exitation at 440/30 nm and emission at 485/20 nm. Readings were every 10 minutes with shaking for 500 seconds before each reading.

For compounds ZP-0089 to ZP-0094, a kinetic ThT experiment was performed in 25 mM Tris-Cl at 37° C. to test the inhibition properties of ZP-0089 to ZP-0094 in Tris-Cl pH 7.4. Each 200 μl sample on the 96 well-plate contained 50 μM ASYN, 100 μM inhibitor and 100 mg/ml PEG along with buffer only and ASYN only controls. Samples were measured in triplicate. ThT fluorescence over time was monitored for 48 hours.

### Inhibition of ASYN Aggregation in PBS

1×PBS was made up from a 10× stock supplied by Gibco at pH 7.4. Sodium azide was added to 0.01% to prevent bacterial growth in the samples. Freeze dried ASYN was made up to 100 μM in PBS and filtered through a 0.22 μM Millex GV filter. A plate containing 100 nmol of each inhibitor was taken from the freezer and allowed to come to room temperature. Each inhibitor was dissolved to a final concentration of 200 μM in 500 μl PBS. 100 μl of ASYN was added to 100 μl inhibitor in each well of a 96 well polypropylene plate (Nunc) to yield a theoretical molar ratio of ASYN:inhibitor 1:2. The plate was covered with a seal and incubated for up to 20 days at 37° C. with shaking on a shaking platform at 1000 rpm.

In order to monitor the extent of aggregation of the sample over time, a 10 μl aliquot of each sample was taken at time=0 and every 1 to 2 days after and frozen at −80° C. to be assayed at the end of the experiment. At the endpoint of the experiment the extent of aggregation was quantified for each sample by measuring thioflavin T (ThT) binding (see above). The final concentration of ASYN diluted in ThT for each sample was 3.45 μM). For each assay, the lag time (the time it takes for the protein to commence aggregation), rate of fibril assembly and final ThT fluorescence, which is related to the amount of conversion was noted and compared to ASYN without the presence of inhibitor.

Two experiments were carried out in the same manner and on different days. FIGS. 3 to 6 show the results for experiments 1 and 2. The endpoint ThT reading is used as a measure of total aggregation undergone by ASYN and the endpoints for reactions containing inhibitor is expressed as a percentage of aggregation of ASYN alone. Results were compared to two control peptides that have been described previously as inhibitors of ASYN aggregation (ASI1 and ASI3) (El-Agnaf, O. M. et al. Faseb J 18, 1315-7 (2004)).

For compounds ZP-0089 to ZP-0094, a kinetic experiment was performed to test the inhibition properties in PBS pH 7.4 as described above. Samples were assayed in triplicate and each sample contained 50 μl of 200 μM ASYN and 50 μl of 400 μM inhibitor was added to 100 μl PBS in a well and mixed to yield a 200 μl sample volume containing 50 μM ASYN and 100 μM inhibitor. Control wells contained either buffer only or ASYN only. FIG. 12 shows the endpoint ThT fluorescence after 258 hours, expressed as a percentage of the fluorescence for ASYN alone.

### Inhibition of ASYN Aggregation in Crowding Agent (PEG 3350).

Four further experiments were carried out using the kinetic ThT assay as described above in the presence of 100 mg/ml PEG 3350 which accelerated the reaction and allowed an assay time of 2 days. All assays were carried out with 50 μM ASYN and a 1:2 molar ratio of ASYN: inhibitor at 37° C. 3 assays were carried out in PBS pH 7.4 (Gibco) and one in 25 mM Tris-Cl pH 7.4 at 37° C.

### Results

A reproducible trend between the two PBS and PEG3500 experiments and many inhibitors show a decrease in the extent of aggregation of more that 20% (See FIGS. 3 to 6).

FIGS. 7 to 9 show a summary of the candidates that showed the highest decrease in endpoint ThT fluorescence in Experiment 1.

Selected peptides from the first round of screening were modified to improve their inhibition properties and add moieties to target the peptides to dopaminergic neurons in brain. Modifications were made to 2 of the designs and synthesized using standard techniques (see Table 3). ZP-0089 and ZP-0090 are modifications of A7 (ZP-0007) and B8 (ZP-0020) with L-DOPA at the N-terminus of the peptide. ZP-0093 has pyroglutamic acid at the N-terminus of A7. FIG. 10 shows the chemical structures of ZP-0089 to ZP-0094.

For inhibitors ZP-0091, ZP-0092 and ZP-0094, ThT fluorescence over time was monitored for 48 hours and the results shown in FIGS. 11 and 12. The decrease in the endpoint ThT reading in Tris-Cl pH 7.4 on average 18±2%. For ZP-0093 the error is too large to be able to make any assumptions at this time and the experiment will be repeated. For inhibitors ZP-0091 to ZP-0094 in PBS, the difference is less marked at an average of 6±2%. However for ZP-0089 and ZP-0090 the inhibitory effect of the peptides is striking in both buffer conditions. ZP-0089 inhibits by >60% and ZP-0090 by >30% compared to ASYN alone. FIG. 13 shows the change in ThT fluorescence over the time of the whole reaction. For ZP-0089 and ZP-0090 the rate of amyloid formation is decreased and there is a large increase in the lag phase seen for ZP-0089. This is the time from the initiation of the reaction to the time when fibrils begin to form. This provides indication that the inhibitor is effecting the nucleation of ASYN and preventing the first steps towards fibril formation from occurring.

Peptides with Modifications for BBB Permeability

Aggregation assays were carried out with 50 μM ASYN and 100 μM inhibitor with 50 mM tris and 150 mM NaCl and 20 μM Thioflavin T. The reaction volume was 200 μL. Each reaction was set up in a 96 well polypropylene plate with ASYN only and buffer only controls. The reactions were incubated at 37 C with shaking for 48 hours and aggregation was monitored by reading thioflavin T fluorescence as described above.

N-methyl phenylalanine (NMePhe) is a blood brain barrier (BBB) transport moiety which has been shown to enhance transport across the BBB. 3 and 4 N-methyl phenylalanine (NMePhe) moieties were coupled to inhibitor ZP-0065 (Ac-kvfgtaa-NH₂) using standard synthetic chemistry to yield the inhibitors ZP-0154 (H-ffff-kvfgtaa-NH2) and ZP-0155 (H-fff-kvfgtaa-NH2) where f is an NMePhe moiety. The chemical structure of ZP-0155 is shown in FIG. 13 below.

The aggregation inhibition properties of ZP-0154 and ZP-0155 were tested in kinetic ThT assays in TBS as described above. The results are shown in FIG. 14.

No significant increase in ThT signal was observed in the timeframe of the assay (48 h) for either peptide ZP-0154 and ZP-0155 compared to ASYN only. This shows that both ZP-0154 and ZP-0155 are effective inhibitors of ASYN aggregation as well as having the potential to be effective at crossing the BBB.

### Interactomer Peptides

A series of peptides were designed to interact with regions 61-66 (EQVTN) and 71-76 (VTGVT). For region 61-65, the peptide Ac-qysvli-NH2 (ZP-0195) was designed to interact and prevent aggregation. Variations of this sequence were also tested where one or more of the amino acids at any given position were substituted with another and variations were made at the N-terminus such as the addition of an extra amino acid and acetylation (ZP-0195 to ZP-0230). For the region 71-75 the peptide Ac-hhviva-NH2 (ZP-0158) was designed to interact and prevent aggregation. Variations of this sequence were also tested where one or more of the amino acids at any give position were substituted with another. All peptides tested were N-terminal acetylated and the 2 histidines at the beginning of the sequence were kept constant in all designs (ZP-0158 to ZP-0194).

All peptides were tested for inhibition of ASYN aggregation in TBS as described above and the kinetic traces were fit using Zyentiafit software which fits the data to a Sigmoidal function f(x)=k+A/(1+exp(−b(t−t0))) from which the lag time, rate of aggregation and total change in ThT fluorescence may be calculated.

Peptides were ranked according to their effectiveness and table 4 shows those sequences chosen for further study. The choice was based on the peptide having more than a 20% increase in lag time and/or more than 20% decrease in ThT fluorescence or aggregation rate.

### Structure Stabilising Peptides

Peptides were designed to reduce or prevent aggregation of α-synuclein by stabilizing secondary structure in the native state. Regions of sequence that have a higher propensity to form a helix were identified using Agadir (EMBL 1997-2002, Lacroix E., Munoz V., Petukhov M. & Serrano, L) as shown in FIG. 16 and peptides were designed to interact with the identified regions of sequence and stabilize secondary structure in that region. The peptides were from 3 to 7 residues in length and contained a mix of polar and non polar residues designed to interact along the face of a specific helix.

A kinetic thioflavin T assay to measure aggregation was set up with 50 uM α-synuclein in TBS and a molar ratio of 1:2 of α-synuclein:inhibitor). Reactions were incubated for 48 hours at 37° C. with shaking and thioflavin T fluorescence was measured every 10 minutes. The kinetic traces were fit using Zyentiafit software which fits the data to a Sigmoidal function f(x)=k+A/(1+exp(−b(t−t0))) from which the lag time, rate of aggregation and total change in ThT fluorescence may be calculated.

Table 5 shows results for a variety of our designed sequences of different length. % differences in lag phase, rate of aggregation and endpoint thioflavin T fluorescence were calculated relative to α-synuclein only. These data shows that interaction with the inhibitors can increase the lag phase by more than 10%. This indicates that by stabilizing secondary structure in α-synuclein, the events leading to nucleation and aggregation may be delayed in-vitro. For the best inhibitor in this series, ZP-0240, a significant decrease in endpoint thioflavin T fluorescence is also observed.

TABLE 1

| | Original | | | Directed |
|---|---|---|---|---|
| | Compound | plate | | against ASYN |
| Name | Name | position | *Sequence | residues |
| ZP-0001 | 1A1 | A1 | Ac-gkmfvdm-NH₂ | 1-7 |
| ZP-0002 | 1A2 | A2 | Ac-gpmfvdm-NH₂ | 1-7 |
| ZP-0003 | 1A3 | A3 | Ac-gkpfvdm-NH₂ | 1-7 |
| ZP-0004 | 2A1 | A4 | Ac-eaaavvg-NH₂ | 14-20 |
| ZP-0005 | 2A2 | A5 | Ac-epaavvg-NH₂ | 14-20 |
| ZP-0006 | 2A3 | A6 | Ac-eapavvg-NH₂ | 14-20 |
| ZP-0007 | 3A1 | A7 | Ac-sgvylvg-NH₂ | 36-42 |
| ZP-0008 | 3A2 | A8 | Ac-spvylvg-NH₂ | 36-42 |
| ZP-0009 | 3A3 | A9 | Ac-sgpylvg-NH₂ | 36-42 |
| ZP-0010 | 4A1 | A10 | Ac-avghvvg-NH₂ | 47-53 |
| ZP-0011 | 4A2 | A11 | Ac-apghvvg-NH₂ | 47-53 |
| ZP-0012 | 4A3 | A12 | Ac-avphvvg-NH₂ | 47-53 |
| ZP-0013 | 4B1 | B1 | Ac-vtavghv-NH₂ | 49-55 |
| ZP-0014 | 4B2 | B2 | Ac-vpavghv-NH₂ | 49-55 |
| ZP-0015 | 4B3 | B3 | Ac-vtpvghv-NH₂ | 49-55 |
| ZP-0016 | 4C1 | B4 | Ac-eavtavg-NH₂ | 51-57 |
| ZP-0017 | 4C2 | B5 | Ac-epvtavg-NH₂ | 51-57 |
| ZP-0018 | 4C3 | B6 | Ac-eaptavg-NH₂ | 51-57 |
| ZP-0019 | 5A1 | B7 | Ac-ggvntvq-NH₂ | 62-68 |
| ZP-0020 | 5A2 | B8 | Ac-gpvntvq-NH₂ | 62-68 |
| ZP-0021 | 5A3 | B9 | Ac-ggpntvq-NH₂ | 62-68 |
| ZP-0022 | 5B1 | B10 | Ac-vaggvnt-NH₂ | 64-70 |
| ZP-0023 | 5B2 | B11 | Ac-vpggvnt-NH₂ | 64-70 |
| ZP-0024 | 5B3 | B12 | Ac-vapgvnt-NH₂ | 64-70 |
| ZP-0025 | 5C1 | C1 | Ac-tvvaggv-NH₂ | 66-72 |
| ZP-0026 | 5C2 | C2 | Ac-tpvaggv-NH₂ | 66-72 |
| ZP-0027 | 5C3 | C3 | Ac-tvpaggv-NH₂ | 66-72 |
| ZP-0028 | 5D1 | C4 | Ac-gtvvagg-NH₂ | 67-73 |
| ZP-0029 | 5D2 | C5 | Ac-gpvvagg-NH₂ | 67-73 |
| ZP-0030 | 5D3 | C6 | Ac-gtpvagg-NH₂ | 67-73 |
| ZP-0031 | 5D4 | C7 | Ac-gtvpagg-NH₂ | 67-73 |
| ZP-0032 | 5E1 | C8 | Ac-vgtvvag-NH₂ | 68-74 |
| ZP-0033 | 5E2 | C9 | Ac-vptvvag-NH₂ | 68-74 |
| ZP-0034 | 5E3 | C10 | Ac-vgpvvag-NH₂ | 68-74 |
| ZP-0035 | 5F1 | C11 | Ac-atvgtvv-NH₂ | 70-76 |
| ZP-0036 | 5F2 | C12 | Ac-apvgtvv-NH₂ | 70-76 |
| ZP-0037 | 5F3 | D1 | Ac-atpgtvv-NH₂ | 70-76 |
| ZP-0038 | 5G1 | D2 | Ac-avatvgt-NH₂ | 72-78 |
| ZP-0039 | 5G2 | D3 | Ac-apatvgt-NH₂ | 72-78 |
| ZP-0040 | 5G3 | D4 | Ac-avptvgt-NH₂ | 72-78 |
| ZP-0041 | 5H1 | D5 | Ac-kqavatv-NH₂ | 74-80 |
| ZP-0042 | 5H2 | D6 | Ac-kpavatv-NH₂ | 74-80 |
| ZP-0043 | 5H3 | D7 | Ac-kqpvatv-NH₂ | 74-80 |
| ZP-0044 | 5I1 | D8 | Ac-vtkqava-NH₂ | 76-82 |
| ZP-0045 | 5I2 | D9 | Ac-vpkqava-NH₂ | 76-82 |
| ZP-0046 | 5I3 | D10 | Ac-vtpqava-NH₂ | 76-82 |
| ZP-0047 | 5J1 | D11 | Ac-gevtkqa-NH₂ | 78-84 |
| ZP-0048 | 5J2 | D12 | Ac-gpvtkqa-NH₂ | 78-84 |
| ZP-0049 | 5J3 | E1 | Ac-geptkqa-NH₂ | 78-84 |
| ZP-0050 | 5K1 | E2 | Ac-gagevtk-NH₂ | 80-86 |
| ZP-0051 | 5K2 | E3 | Ac-gpgevtk-NH₂ | 80-86 |
| ZP-0052 | 5K3 | E4 | Ac-gapevtk-NH₂ | 80-86 |
| ZP-0053 | 5L1 | E5 | Ac-isgagev-NH₂ | 82-88 |
| ZP-0054 | 5L2 | E6 | Ac-ipgagev-NH₂ | 82-88 |
| ZP-0055 | 5L3 | E7 | Ac-ispagev-NH₂ | 82-88 |
| ZP-0056 | 5M1 | E8 | Ac-aaisgag-NH₂ | 84-90 |
| ZP-0057 | 5M2 | E9 | Ac-apisgag-NH₂ | 84-90 |
| ZP-0058 | 5M3 | E10 | Ac-aapsgag-NH₂ | 84-90 |
| ZP-0059 | 5N1 | E11 | Ac-taaaisg-NH₂ | 86-92 |
| ZP-0060 | 5N2 | E12 | Ac-tpaaisg-NH₂ | 86-92 |
| ZP-0061 | 5N3 | F1 | Ac-tapaisg-NH₂ | 86-92 |
| ZP-0062 | 5O1 | F2 | Ac-fgtaaai-NH₂ | 88-94 |
| ZP-0063 | 5O2 | F3 | Ac-fptaaai-NH₂ | 88-94 |
| ZP-0064 | 5O3 | F4 | Ac-fgpaaai-NH₂ | 88-94 |
| ZP-0065 | 5P1 | F5 | Ac-kvfgtaa-NH₂ | 90-96 |
| ZP-0066 | 5P2 | F6 | Ac-kpfgtaa-NH₂ | 90-96 |
| ZP-0067 | 5P3 | F7 | Ac-kvpgtaa-NH₂ | 90-96 |
| ZP-0068 | 6C1 | F12 | Ac-sgtvvags-NH₂ | 68-73 |
| ZP-0069 | 6C2 | G1 | Ac-sptvvags-NH₂ | 68-73 |
| ZP-0070 | 6C3 | G2 | Ac-sgpvvags-NH₂ | 68-73 |
| ZP-0071 | 6E1 | G4 | Ac-sgtvvaggs-NH₂ | 69-73 |
| ZP-0072 | 6E2 | G5 | Ac-sptvvaggs-NH₂ | 69-73 |
| ZP-0073 | 6E3 | G6 | Ac-sgpvvaggs-NH₂ | 69-73 |
| ZP-0074 | 7A1 | G7 | Ac-vpapghv-NH₂ | 49-57 |
| ZP-0075 | 7A2 | G8 | Ac-vtpvphv-NH₂ | 49-57 |
| ZP-0076 | 7A3 | G9 | Ac-vtapgpv-NH₂ | 49-57 |
| ZP-0077 | 7A4 | G10 | Ac-vpavphv-NH₂ | 49-57 |
| ZP-0078 | 7A5 | G11 | Ac-vtpvgpv-NH₂ | 49-57 |
| ZP-0079 | 7A6 | G12 | Ac-vpapgpv-NH₂ | 49-57 |
| ZP-0080 | 7B1 | H1 | Ac-gpvpagg-NH₂ | 67-73 |
| ZP-0081 | 7B2 | H2 | Ac-gtpvpgg-NH₂ | 67-73 |
| ZP-0082 | 7B3 | H3 | Ac-gtvpapg-NH₂ | 67-73 |
| ZP-0083 | 7B4 | H4 | Ac-gpvvpgg-NH₂ | 67-73 |
| ZP-0084 | 7B5 | H5 | Ac-gtpvapg-NH₂ | 67-73 |
| ZP-0085 | 7B6 | H6 | Ac-gpvpapg-NH₂ | 67-73 |
| ZP-0086 | 8A1 | H7 | Ac-vgektkg-NH₂ | 31-37 (NC) |
| ZP-0087 | 8B1 | H8 | Ac-aendpdv-NH₂ | 118-124 (NC) |

Ac = acetylated N-terminus
NC = negative control
Lower case sequence = D-amino acids

TABLE 2

| | Original | | | Directed |
|---|---|---|---|---|
| | Compound | plate | | against ASYN |
| Name | Name | position | Sequence | residues |
| ZP-0001 | 1A1 | A1 | Ac-gkmfvdm-NH₂ | 1-7 |
| ZP-0007 | 3A1 | A7 | Ac-sgvylvg-NH₂ | 36-42 |
| ZP-0009 | 3A3 | A9 | Ac-sgpylvg-NH₂ | 36-42 |
| ZP-0013 | 4B1 | B1 | Ac-vtavghv-NH₂ | 49-55 |
| ZP-0024 | 5B3 | B12 | Ac-vapgvnt-NH₂ | 64-70 |
| ZP-0025 | 5C1 | C1 | Ac-tvvaggv-NH₂ | 66-72 |
| ZP-0035 | 5F1 | C11 | Ac-atvgtvv-NH₂ | 70-76 |
| ZP-0052 | 5K3 | E4 | Ac-gapevtk-NH₂ | 80-86 |
| ZP-0059 | 5N1 | E11 | Ac-taaaisg-NH₂ | 86-92 |
| ZP-0060 | 5N2 | E12 | Ac-tpaaisg-NH₂ | 86-92 |
| ZP-0061 | 5N3 | F1 | Ac-tapaisg-NH₂ | 86-92 |
| ZP-0062 | 5O1 | F2 | Ac-fgtaaai-NH₂ | 88-94 |
| ZP-0063 | 5O2 | F3 | Ac-fptaaai-NH₂ | 88-94 |
| ZP-0064 | 5O3 | F4 | Ac-fgpaaai-NH₂ | 88-94 |
| ZP-0065 | 5P1 | F5 | Ac-kvfgtaa-NH₂ | 90-96 |
| ZP-0066 | 5P2 | F6 | Ac-kpfgtaa-NH₂ | 90-96 |
| ZP-0088 | 5PX | *FX | Ac-kvfptaa-NH₂ | 90-96 |
| ZP-0069 | 6C2 | G1 | Ac-sptvvags-NH₂ | 68-73 |

TABLE 3

| Zyentia | Peptide | Parental | |
|---|---|---|---|
| Code | Code | peptide | Sequence and Modification |
| ZP-0089 | EG4 | ZP-0007 | ¹L-DOPA-sgvylvg-NH₂ |
| ZP-0090 | EG5 | ZP-0020 | ¹L-DOPA-gpvntvq-NH₂ |
| ZP-0091 | EG6 | ZP-0007 | Ac-sgvylvg-COOH |
| ZP-0092 | EG7 | ZP-0007 | NH2-sgvylvg-COOH |
| ZP-0093 | EG8 | ZP-0007 | ²pGlu-sgvylvg-NH₂ |
| ZP-0094 | EG9 | ZP-0007 | NH2-sgvylvg-NH₂ |
| ZP-0154 | EG15 | ZP-0065 | ³NMP-NMP-NMP-NMP-kvfgtaa-NH2 |
| ZP-0155 | EG16 | ZP-0065 | ³NMP-NMP-NMP-kvfgtaa-NH2 |

¹L-DOPA = levodopa or 3,4-dihydroxy-L-phenylalanine
²pGlu = pyroglutamic acid
³NMP = N-methyl phenylalanine

TABLE 4

| | | % decrease | % decrease | % increase |
|---|---|---|---|---|
| Zyentia | | in ThT | in rate of | in Lag |
| Code | Sequence | fluorescence | aggregation | phase |
| ZP-0158 | Ac-hhviva-NH2 | 15.1 | 18.4 | 32.5 |
| ZP-0159 | Ac-hhvvva-NH2 | 19.8 | 18.6 | 36.6 |
| ZP-0160 | Ac-hhvlva-NH2 | 8.8 | 10.5 | 36.5 |
| ZP-0161 | Ac-hhvkva-NH2 | −5.9 | 2.3 | 19.7 |
| ZP-0162 | Ac-hhveva-NH2 | 6.3 | 6.6 | 33.4 |
| ZP-0164 | Ac-hpviva-NH2 | 3.7 | −2.3 | 43.0 |
| ZP-0168 | Ac-hhvivp-NH2 | −8.6 | −5.4 | 29.8 |
| ZP-0169 | Ac-hhvivv-NH2 | −0.5 | −13.3 | 26.1 |
| ZP-0170 | Ac-hhvivt-NH2 | 24.5 | 29.3 | 29.4 |
| ZP-0171 | Ac-hhvivy-NH2 | 13.9 | 13.2 | 43.6 |
| ZP-0172 | Ac-hhvivw-NH2 | −1.7 | 6.9 | 28.1 |
| ZP-0175 | Ac-hhtivv-NH2 | 21.9 | 16.8 | 89.7 |
| ZP-0179 | Ac-hhtivk-NH2 | 0.6 | −0.6 | 30.4 |
| ZP-0180 | Ac-hhtvva-NH2 | 19.8 | 18.4 | 30.3 |
| ZP-0181 | Ac-hhtlva-NH2 | 22.8 | −1.2 | 29.4 |
| ZP-0186 | Ac-hhtlvv-NH2 | 30.7 | 29.7 | 16.1 |
| ZP-0193 | Ac-hhtevy-NH2 | 8.7 | −15.8 | 31.4 |
| ZP-0194 | Ac-hhttvy-NH2 | 11.6 | 7.7 | 38.8 |
| ZP-0202 | Ac-qykvli-NH2 | 53.5 | 54.5 | −28.8 |
| ZP-0204 | Ac-qysvpi-NH2 | 1.4 | −5.6 | 23.6 |
| ZP-0205 | Ac-qyspli-NH2 | 16.6 | −11.9 | 30.6 |
| ZP-0206 | Ac-qypvli-NH2 | 3.3 | 8.4 | 25.9 |
| ZP-0207 | Ac-qpsvli-NH2 | 5.5 | 3.6 | 29.7 |
| ZP-0212 | Ac-rysvli-NH2 | 51.1 | 44.9 | −14.4 |
| ZP-0213 | Ac-ekysvli-NH2 | 25.6 | 12.9 | 29.4 |
| ZP-0214 | Ac-drysvli-NH2 | 20.4 | 4.8 | −12.6 |
| ZP-0215 | NH3-qysvli-NH2 | 27.6 | 17.5 | 3.1 |
| ZP-0221 | NH3-qytvli-NH2 | 24.7 | 22.8 | −22.4 |
| ZP-0222 | NH3-qykvli-NH2 | 42.3 | 34.3 | 7.7 |
| ZP-0228 | NH3-pysvli-NH2 | 40.8 | 46.2 | 38.2 |
| ZP-0229 | NH3-qysvlv-NH2 | 17.8 | 24.3 | 33.5 |
| ZP-0230 | NH3-qysvlt-NH2 | 10.9 | 15.9 | 32.4 |

TABLE 5

| | | % decrease | % decrease | % increase |
|---|---|---|---|---|
| Zyentia | | in ThT | in rate of | in Lag |
| Code | Sequence | fluorescence | aggregation | phase |
| ZP-0231 | Ac-kgegk-NH2 | 4.3 | 2.7 | 11.2 |
| ZP-0240 | Ac-rdr-NH2 | 18.5 | −0.6 | 23.4 |
| ZP-0241 | Ac-egkgegk-NH2 | 6.1 | 10.9 | 16.6 |
| ZP-0247 | Ac-rgdgd-NH2 | 12.2 | 6.7 | 10.7 |

1. A peptide consisting of four to ten D-amino acids having the reverse sequence of a contiguous amino acid sequence within the region between residues 86-96 of α-synuclein.

2.-7. (canceled)

8. The peptide according to claim 1, consisting of a sequence of D-amino acids selected from the group consisting of: taaaisg, fgtaaai and kvfgtaa.

9. A peptide consisting of the D-amino acid sequence of the peptide according to claim 8 with the addition, deletion or substitution of 1 to 3 D-amino acid residues.

10. The peptide according to claim 9 wherein one or more D-amino acid residues of said peptide is replaced by proline.

11.-30. (canceled)

31. The peptide according to claim 1, which is linked to one or more coupling partners.

32. The peptide according to claim 31 wherein the coupling partner is a protecting group or a dopaminergic neuron targeting moiety.

33. The peptide according to claim 32 wherein the protecting group is an acetyl, amide or 3 to 20 carbon alkyl group.

34.-35. (canceled)

36. The peptide according to claim 33 wherein the dopaminergic neuron targeting moiety is a dopamine analogue or DOPA agonist.

37. The peptide according to claim 36 wherein the dopaminergic neuron targeting moiety is L-DOPA or pyroglutamic acid.

38. The peptide according to claim 11 wherein the coupling partner is a Blood Brain Barrier (BBB) transport moiety.

39. The peptide according to claim 38 wherein the Blood Brain Barrier (BBB) transport moiety is N-methyl phenylalanine (NMePhe).

40. The peptide according to claim 39 comprising 1 to 5 N-methyl phenylalanine (NMePhe) moieties.

41. (canceled)

42. A pharmaceutical composition comprising one or more peptides according to claim 1 and a pharmaceutically acceptable excipient.

43.-47. (canceled)

48. A method of treatment of α-synucleinopathy in an individual comprising administering the peptide according to claim 1 to said individual.

49. A method of treatment of α-synucleinopathy in an individual comprising administering the composition according to claim 42 to said individual.


SEQUENCE LISTING (133 sequences)

SEQ ID NO: 1 - protein, 7 aa, Artificial sequence
  Synthetic peptide consisting of a sequence of D-amino acids
GKMFVDM

SEQ ID NO: 2 - protein, 7 aa, Artificial sequence
  Synthetic peptide consisting of a sequence of D-amino acids
SGVYLVG

SEQ ID NO: 3 - protein, 7 aa, Artificial sequence
  Synthetic peptide consisting of a sequence of D-amino acids
VTAVGHV

SEQ ID NO: 4 - protein, 7 aa, Artificial sequence
  Synthetic peptide consisting of a sequence of D-amino acids
TVVAGGV

SEQ ID NO: 5 - protein, 7 aa, Artificial sequence
  Synthetic peptide consisting of a sequence of D-amino acids
ATVGTVV

SEQ ID NO: 6 - protein, 7 aa, Artificial sequence
  Synthetic peptide consisting of a sequence of D-amino acids
TAAAISG

SEQ ID NO: 7 - protein, 7 aa, Artificial sequence
  Synthetic peptide consisting of a sequence of D-amino acids
FGTAAAI

SEQ ID NO: 8 - protein, 7 aa, Artificial sequence
  Synthetic peptide consisting of a sequence of D-amino acids
KVFGTAA

SEQ ID NO: 9 - protein, 4 aa, Artificial sequence
  Reverse sequence of a contiguous amino acid sequence from human alpha-synuclein
TVVA

SEQ ID NO: 10 - protein, 4 aa, Artificial sequence
  Reverse sequence of a contiguous amino acid sequence from human alpha-synuclein
AAAI

SEQ ID NO: 11 - protein, 5 aa, Homo sapiens
EQVTN

SEQ ID NO: 12 - protein, 6 aa, Artificial sequence
  Synthetic peptide consisting of a sequence of D-amino acids
QYSVLI

SEQ ID NO: 13 - protein, 6 aa, Artificial sequence
  Synthetic peptide consisting of a sequence of D-amino acids
QYKVLI

SEQ ID NO: 14 - protein, 6 aa, Artificial sequence
  Synthetic peptide consisting of a sequence of D-amino acids
QYSVPI

SEQ ID NO: 15 - protein, 6 aa, Artificial sequence
  Synthetic peptide consisting of a sequence of D-amino acids
QYSPLI

SEQ ID NO: 16 - protein, 6 aa, Artificial sequence
  Synthetic peptide consisting of a sequence of D-amino acids
QYPVLI

SEQ ID NO: 17 - protein, 6 aa, Artificial sequence
  Synthetic peptide consisting of a sequence of D-amino acids
RYSVLI

SEQ ID NO: 18 - protein, 6 aa, Artificial sequence
  Synthetic peptide consisting of a sequence of D-amino acids
QYTVLI

SEQ ID NO: 19 - protein, 6 aa, Artificial sequence
  Synthetic peptide consisting of a sequence of D-amino acids
PYSVLI

SEQ ID NO: 20 - protein, 6 aa, Artificial sequence
  Synthetic peptide consisting of a sequence of D-amino acids
QYSVLV

SEQ ID NO: 21 - protein, 7 aa, Artificial sequence
  Synthetic peptide consisting of a sequence of D-amino acids
EKYSVLI

SEQ ID NO: 22 - protein, 7 aa, Artificial sequence
  Synthetic peptide consisting of a sequence of D-amino acids
DRYSVLI

SEQ ID NO: 23 - protein, 5 aa, Homo sapiens
VTGVT

SEQ ID NO: 24 - protein, 6 aa, Artificial sequence
  Synthetic peptide consisting of a sequence of D-amino acids
HHVIVA

SEQ ID NO: 25 - protein, 6 aa, Artificial sequence
  Synthetic peptide consisting of a sequence of D-amino acids
HHVVVA

SEQ ID NO: 26 - protein, 6 aa, Artificial sequence
  Synthetic peptide consisting of a sequence of D-amino acids
HHVLVA

SEQ ID NO: 27 - protein, 6 aa, Artificial sequence
  Synthetic peptide consisting of a sequence of D-amino acids
HHVKVA

SEQ ID NO: 28 - protein, 6 aa, Artificial sequence
  Synthetic peptide consisting of a sequence of D-amino acids
HHVEVA

SEQ ID NO: 29 - protein, 6 aa, Artificial sequence
  Synthetic peptide consisting of a sequence of D-amino acids
HPVIVA

SEQ ID NO: 30 - protein, 6 aa, Artificial sequence
  Synthetic peptide consisting of a sequence of D-amino acids
HHVIVP

SEQ ID NO: 31 - protein, 6 aa, Artificial sequence
  Synthetic peptide consisting of a sequence of D-amino acids
HHVIVV

SEQ ID NO: 32 - protein, 6 aa, Artificial sequence
  Synthetic peptide consisting of a sequence of D-amino acids
HHVIVT

SEQ ID NO: 33 - protein, 6 aa, Artificial sequence
  Synthetic peptide consisting of a sequence of D-amino acids
HHVIVY

SEQ ID NO: 34 - protein, 6 aa, Artificial sequence
  Synthetic peptide consisting of a sequence of D-amino acids
HHVIVW

SEQ ID NO: 35 - protein, 6 aa, Artificial sequence
  Synthetic peptide consisting of a sequence of D-amino acids
HHTIVV

SEQ ID NO: 36 - protein, 6 aa, Artificial sequence
  Synthetic peptide consisting of a sequence of D-amino acids
HHTIVK

SEQ ID NO: 37 - protein, 6 aa, Artificial sequence
  Synthetic peptide consisting of a sequence of D-amino acids
HHTVVA

SEQ ID NO: 38 - protein, 6 aa, Artificial sequence
  Synthetic peptide consisting of a sequence of D-amino acids
HHTLVA

SEQ ID NO: 39 - protein, 6 aa, Artificial sequence
  Synthetic peptide consisting of a sequence of D-amino acids
HHTLVV

SEQ ID NO: 40 - protein, 6 aa, Artificial sequence
  Synthetic peptide consisting of a sequence of D-amino acids
HHTEVY

SEQ ID NO: 41 - protein, 6 aa, Artificial sequence
  Synthetic peptide consisting of a sequence of D-amino acids
HHTTVY

SEQ ID NO: 42 - protein, 5 aa, Artificial sequence
  Synthetic peptide consisting of a sequence of D-amino acids
KGEGK

SEQ ID NO: 43 - protein, 7 aa, Artificial sequence
  Synthetic peptide consisting of a sequence of D-amino acids
EGKGEGK

SEQ ID NO: 44 - protein, 5 aa, Artificial sequence
  Synthetic peptide consisting of a sequence of D-amino acids
RGDGD

SEQ ID NO: 45 - protein, 7 aa, Artificial sequence
  Synthetic peptide consisting of a sequence of D-amino acids
GAPEVTK

SEQ ID NO: 46 - protein, 7 aa, Artificial sequence
  Synthetic peptide consisting of a sequence of D-amino acids
TPAAISG

SEQ ID NO: 47 - protein, 7 aa, Artificial sequence
  Synthetic peptide consisting of a sequence of D-amino acids
TAPAISG

SEQ ID NO: 48 - protein, 7 aa, Artificial sequence
  Synthetic peptide consisting of a sequence of D-amino acids
FPTAAAI

SEQ ID NO: 49 - protein, 7 aa, Artificial sequence
  Synthetic peptide consisting of a sequence of D-amino acids
KPFGTAA

SEQ ID NO: 50 - protein, 7 aa, Artificial sequence
  Synthetic peptide consisting of a sequence of D-amino acids
KVFPTAA

SEQ ID NO: 51 - protein, 8 aa, Artificial sequence
  Synthetic peptide consisting of a sequence of D-amino acids
SPTVVAGS

SEQ ID NO: 52 - protein, 7 aa, Artificial sequence
  Synthetic peptide consisting of a sequence of D-amino acids
GPVNTVQ

SEQ ID NO: 53 - protein, 11 aa, Artificial sequence
  Synthetic peptide comprising a sequence of D-amino acids
XXXXKVFGTAA

SEQ ID NO: 54 - protein, 10 aa, Artificial sequence
  Synthetic peptide comprising a sequence of D-amino acids
XXXKVFGTAA

SEQ ID NO: 55 - protein, 7 aa, Artificial sequence
  Synthetic peptide consisting of a sequence of D-amino acids
GPMFVDM

SEQ ID NO: 56 - protein, 7 aa, Artificial sequence
  Synthetic peptide consisting of a sequence of D-amino acids
GKPFVDM

SEQ ID NO: 57 - protein, 7 aa, Artificial sequence
  Synthetic peptide consisting of a sequence of D-amino acids
EAAAVVG

SEQ ID NO: 58 - protein, 7 aa, Artificial sequence
  Synthetic peptide consisting of a sequence of D-amino acids
EPAAVVG

SEQ ID NO: 59 - protein, 7 aa, Artificial sequence
  Synthetic peptide consisting of a sequence of D-amino acids
EAPAVVG

SEQ ID NO: 60 - protein, 7 aa, Artificial sequence
  Synthetic peptide consisting of a sequence of D-amino acids
SPVYLVG

SEQ ID NO: 61 - protein, 7 aa, Artificial sequence
  Synthetic peptide consisting of a sequence of D-amino acids
SGPYLVG

SEQ ID NO: 62 - protein, 7 aa, Artificial sequence
  Synthetic peptide consisting of a sequence of D-amino acids
AVGHVVG

SEQ ID NO: 63 - protein, 7 aa, Artificial sequence
  Synthetic peptide consisting of a sequence of D-amino acids
APGHVVG

SEQ ID NO: 64 - protein, 7 aa, Artificial sequence
  Synthetic peptide consisting of a sequence of D-amino acids
AVPHVVG

SEQ ID NO: 65 - protein, 7 aa, Artificial sequence
  Synthetic peptide consisting of a sequence of D-amino acids
VPAVGHV

SEQ ID NO: 66 - protein, 7 aa, Artificial sequence
  Synthetic peptide consisting of a sequence of D-amino acids
VTPVGHV

SEQ ID NO: 67 - protein, 7 aa, Artificial sequence
  Synthetic peptide consisting of a sequence of D-amino acids
EAVTAVG

SEQ ID NO: 68 - protein, 7 aa, Artificial sequence
  Synthetic peptide consisting of a sequence of D-amino acids
EPVTAVG

SEQ ID NO: 69 - protein, 7 aa, Artificial sequence
  Synthetic peptide consisting of a sequence of D-amino acids
EAPTAVG

SEQ ID NO: 70 - protein, 7 aa, Artificial sequence
  Synthetic peptide consisting of a sequence of D-amino acids
GGVNTVQ

SEQ ID NO: 71 - protein, 7 aa, Artificial sequence
  Synthetic peptide consisting of a sequence of D-amino acids
GPVNTVQ

SEQ ID NO: 72 - protein, 7 aa, Artificial sequence
  Synthetic peptide consisting of a sequence of D-amino acids
GGPNTVQ

SEQ ID NO: 73 - protein, 7 aa, Artificial sequence
  Synthetic peptide consisting of a sequence of D-amino acids
VAGGVNT

SEQ ID NO: 74 - protein, 7 aa, Artificial sequence
  Synthetic peptide consisting of a sequence of D-amino acids
VPGGVNT

SEQ ID NO: 75 - protein, 7 aa, Artificial sequence
  Synthetic peptide consisting of a sequence of D-amino acids
VAPGVNT

SEQ ID NO: 76 - protein, 7 aa, Artificial sequence
  Synthetic peptide consisting of a sequence of D-amino acids
TPVAGGV

SEQ ID NO: 77 - protein, 7 aa, Artificial sequence
  Synthetic peptide consisting of a sequence of D-amino acids
TVPAGGV

SEQ ID NO: 78 - protein, 7 aa, Artificial sequence
  Synthetic peptide consisting of a sequence of D-amino acids
GTVVAGG

SEQ ID NO: 79 - protein, 7 aa, Artificial sequence
  Synthetic peptide consisting of a sequence of D-amino acids
GPVVAGG

SEQ ID NO: 80 - protein, 7 aa, Artificial sequence
  Synthetic peptide consisting of a sequence of D-amino acids
GTPVAGG

SEQ ID NO: 81 - protein, 7 aa, Artificial sequence
  Synthetic peptide consisting of a sequence of D-amino acids
GTVPAGG

SEQ ID NO: 82 - protein, 7 aa, Artificial sequence
  Synthetic peptide consisting of a sequence of D-amino acids
VGTVVAG

SEQ ID NO: 83 - protein, 7 aa, Artificial sequence
  Synthetic peptide consisting of a sequence of D-amino acids
VPTVVAG

SEQ ID NO: 84 - protein, 7 aa, Artificial sequence
  Synthetic peptide consisting of a sequence of D-amino acids
VGPVVAG

SEQ ID NO: 85 - protein, 7 aa, Artificial sequence
  Synthetic peptide consisting of a sequence of D-amino acids
APVGTVV

SEQ ID NO: 86 - protein, 7 aa, Artificial sequence
  Synthetic peptide consisting of a sequence of D-amino acids
ATPGTVV

SEQ ID NO: 87 - protein, 7 aa, Artificial sequence
  Synthetic peptide consisting of a sequence of D-amino acids
AVATVGT

SEQ ID NO: 88 - protein, 7 aa, Artificial sequence
  Synthetic peptide consisting of a sequence of D-amino acids
APATVGT

SEQ ID NO: 89 - protein, 7 aa, Artificial sequence
  Synthetic peptide consisting of a sequence of D-amino acids
AVPTVGT

SEQ ID NO: 90 - protein, 7 aa, Artificial sequence
  Synthetic peptide consisting of a sequence of D-amino acids
KQAVATV

SEQ ID NO: 91 - protein, 7 aa, Artificial sequence
  Synthetic peptide consisting of a sequence of D-amino acids
KPAVATV

SEQ ID NO: 92 - protein, 7 aa, Artificial sequence
  Synthetic peptide consisting of a sequence of D-amino acids
KQPVATV

SEQ ID NO: 93 - protein, 7 aa, Artificial sequence
  Synthetic peptide consisting of a sequence of D-amino acids
VTKQAVA

SEQ ID NO: 94 - protein, 7 aa, Artificial sequence
  Synthetic peptide consisting of a sequence of D-amino acids
VPKQAVA

SEQ ID NO: 95 - protein, 7 aa, Artificial sequence
  Synthetic peptide consisting of a sequence of D-amino acids
VTPQAVA

SEQ ID NO: 96 - protein, 7 aa, Artificial sequence
  Synthetic peptide consisting of a sequence of D-amino acids
GEVTKQA

SEQ ID NO: 97 - protein, 7 aa, Artificial sequence
  Synthetic peptide consisting of a sequence of D-amino acids
GPVTKQA

SEQ ID NO: 98 - protein, 7 aa, Artificial sequence
  Synthetic peptide consisting of a sequence of D-amino acids
GEPTKQA

SEQ ID NO: 99 - protein, 7 aa, Artificial sequence
  Synthetic peptide consisting of a sequence of D-amino acids
GAGEVTK

SEQ ID NO: 100 - protein, 7 aa, Artificial sequence
  Synthetic peptide consisting of a sequence of D-amino acids
GPGEVTK

SEQ ID NO: 101 - protein, 7 aa, Artificial sequence
  Synthetic peptide consisting of a sequence of D-amino acids
ISGAGEV

SEQ ID NO: 102 - protein, 7 aa, Artificial sequence
  Synthetic peptide consisting of a sequence of D-amino acids
IPGAGEV

SEQ ID NO: 103 - protein, 7 aa, Artificial sequence
  Synthetic peptide consisting of a sequence of D-amino acids
ISPAGEV

SEQ ID NO: 104 - protein, 7 aa, Artificial sequence
  Synthetic peptide consisting of a sequence of D-amino acids
AAISGAG

SEQ ID NO: 105 - protein, 7 aa, Artificial sequence
  Synthetic peptide consisting of a sequence of D-amino acids
APISGAG

SEQ ID NO: 106 - protein, 7 aa, Artificial sequence
  Synthetic peptide consisting of a sequence of D-amino acids
AAPSGAG

SEQ ID NO: 107 - protein, 7 aa, Artificial sequence
  Synthetic peptide consisting of a sequence of D-amino acids
FGPAAAI

SEQ ID NO: 108 - protein, 7 aa, Artificial sequence
  Synthetic peptide consisting of a sequence of D-amino acids
KVPGTAA

SEQ ID NO: 109 - protein, 8 aa, Artificial sequence
  Synthetic peptide consisting of a sequence of D-amino acids
SGTVVAGS

SEQ ID NO: 110 - protein, 8 aa, Artificial sequence
  Synthetic peptide consisting of a sequence of D-amino acids
SGPVVAGS

SEQ ID NO: 111 - protein, 9 aa, Artificial sequence
  Synthetic peptide consisting of a sequence of D-amino acids
SGTVVAGGS

SEQ ID NO: 112 - protein, 9 aa, Artificial sequence
  Synthetic peptide consisting of a sequence of D-amino acids
SPTVVAGGS

SEQ ID NO: 113 - protein, 9 aa, Artificial sequence
  Synthetic peptide consisting of a sequence of D-amino acids
SGPVVAGGS

SEQ ID NO: 114 - protein, 7 aa, Artificial sequence
  Synthetic peptide consisting of a sequence of D-amino acids
VPAPGHV

SEQ ID NO: 115 - protein, 7 aa, Artificial sequence
  Synthetic peptide consisting of a sequence of D-amino acids
VTPVPHV

SEQ ID NO: 116 - protein, 7 aa, Artificial sequence
  Synthetic peptide consisting of a sequence of D-amino acids
VTAPGPV

SEQ ID NO: 117 - protein, 7 aa, Artificial sequence
  Synthetic peptide consisting of a sequence of D-amino acids
VPAVPHV

SEQ ID NO: 118 - protein, 7 aa, Artificial sequence
  Synthetic peptide consisting of a sequence of D-amino acids
VTPVGPV

SEQ ID NO: 119 - protein, 7 aa, Artificial sequence
  Synthetic peptide consisting of a sequence of D-amino acids
VPAPGPV

SEQ ID NO: 120 - protein, 7 aa, Artificial sequence
  Synthetic peptide consisting of a sequence of D-amino acids
GPVPAGG

SEQ ID NO: 121 - protein, 7 aa, Artificial sequence
  Synthetic peptide consisting of a sequence of D-amino acids
GTPVPGG

SEQ ID NO: 122 - protein, 7 aa, Artificial sequence
  Synthetic peptide consisting of a sequence of D-amino acids
GTVPAPG

SEQ ID NO: 123 - protein, 7 aa, Artificial sequence
  Synthetic peptide consisting of a sequence of D-amino acids
GPVVPGG

SEQ ID NO: 124 - protein, 7 aa, Artificial sequence
  Synthetic peptide consisting of a sequence of D-amino acids
GTPVAPG

SEQ ID NO: 125 - protein, 7 aa, Artificial sequence
  Synthetic peptide consisting of a sequence of D-amino acids
GPVPAPG

SEQ ID NO: 126 - protein, 7 aa, Artificial sequence
  Synthetic peptide consisting of a sequence of D-amino acids
VGEKTKG

SEQ ID NO: 127 - protein, 7 aa, Artificial sequence
  Synthetic peptide consisting of a sequence of D-amino acids
AENDPDV

SEQ ID NO: 128 - protein, 8 aa, Artificial sequence
  Synthetic peptide comprising a sequence of D-amino acids
XSGVYLVG

SEQ ID NO: 129 - protein, 8 aa, Artificial sequence
  Synthetic peptide comprising a sequence of D-amino acids
XGPVNTVQ

SEQ ID NO: 130 - protein, 8 aa, Artificial sequence
  Synthetic peptide comprising a sequence of D-amino acids
XSGVYLVG

SEQ ID NO: 131 - protein, 6 aa, Artificial sequence
  Synthetic peptide consisting of a sequence of D-amino acids
QPSVLI

SEQ ID NO: 132 - protein, 6 aa, Artificial sequence
  Synthetic peptide consisting of a sequence of D-amino acids
QYKVLI

SEQ ID NO: 133 - protein, 6 aa, Artificial sequence
  Synthetic peptide consisting of a sequence of D-amino acids
QYSVLT